(19) United States
(12) Patent Application Publication
(10) Pub. No.: US 2005/0147652 A1
(43) Pub. Date: Jul. 7, 2005

(54) Cell delivery system

(76) Inventors: Jane Tjia Atkins, Woburn, MA (US); Michael David Leek, Rixton (GB); Paul David Kemp, Cheshire (GB); Richard Gregory Wolowacz, Derbyshire (GB); Jeffrey Keeler Teumer, Brookline, MA (US)

Correspondence Address: EDWARDS & ANGELL, LLP, P.O. BOX 55874, BOSTON, MA, 02205, US

(21) Appl. No.: 10/504,789

(22) PCT Filed: Feb. 10, 2003
(86) PCT No.: PCT/GB03/00587

Related U.S. Application Data
(63) Continuation-in-part of application No. 10/074,221, filed on Feb. 12, 2002, now abandoned.

(51) Int. Cl. A61K 7/06; A61K 9/70

(52) U.S. Cl. 424/443; 424/70.1

(57) ABSTRACT

Baldness or hair loss has been treated using pharmaceutical drugs and/or hair transplant surgery. The present invention relates to an improved method for treating baldness. In one aspect, there is provided a method for inducing hair follicle formation in a dermal layer lying beneath an outer skin surface, comprising delivering inductive dermal sheath cells and/or inductive dermal papilla cells directly into the dermal layer using a controlled delivery device. Delivery of the inductive cells need not be into pre-existing pores. Use of a controlled delivery device for the treatment of baldness is a further aspect of the invention.

The present invention relates to delivery of inductive cells for generation of new organs, for example hair follicles.

Mammalian skin is composed of two layers, an outer layer called the epidermis and an inner layer called the dermis. The epidermis is several cell layers thick, is comprised of mainly keratinocyte cells, and has an external layer of dead cells that are constantly shed from the surface and replaced from below by a basal layer of cells, the stratum germinativum. The dermis comprises a network of collagenous extracellular material, elastic fibres, blood vessels, nerves and hair follicles with associated sebaceous glands.

During embryogenesis, the establishment of a dermal papilla is vital to the development of hair follicles and associated modified structures like sebaceous glands. The dermal papilla is a group of specialised dermal fibroblast cells, derived from the embryonic mesoderm. These dermal papilla cells begin to aggregate in the dermis just below the epidermis. Above the dermal papilla an epidermal plug, or peg, of cells develops and proliferates growing into the dermis towards the dermal papilla. The mesoderm-derived dermal papilla and the ectoderm-derived epidermal plug communicate via molecular signals with the result of further proliferation of epidermal matrix cells and differentiation into the various sheath and hair fibre structures. Thus the development of a hair follicle requires a continuum through induction, initiation, elongation and differentiation stages.

A mature hair follicle comprises a bulb containing the dermal papilla cells, a hair shaft extending from the bulb through to the exterior of the epidermis, and a dermal sheath which provides an external covering of tissue around the bulb and along the length of the follicle. The hair follicle extends down through the dermis, a hypodermis (a loose layer of connective tissue below the dermis), and a fat or adipose layer. In adults, molecular signals between the dermal papilla and the epidermal component of a follicle cause the hair to enter an active (anagen) growth phase from an inactive (telogen) phase.

Baldness (known medically as alopecia) is defined as the absence of hair from an area of the body, especially where hair normally exists. Baldness can exist or arise for several reasons. Lack of hair can be caused by the non-presence of hair follicles, for example for genetic reasons. Hair loss can be caused by destruction (for example scarring), disease, infection and/or disruption of the natural hair growth cycle (for example, due to insensitivity to hormones).

For cosmetic and/or aesthetic reasons, several methods for treating baldness have been attempted. One approach has been to use pharmaceutical drugs (such as Minoxidil [RTM; Rogaine, Upjohn] and Finasteride [RTM; Propecia, Merck]). However, pharmaceuticals have achieved limited success in restoring natural hair growth.

Another approach, particularly for hair loss, has been hair transplantation, for example where tissue comprising hair follicles is transplanted from a site where the hair follicles are insensitive to dihydroxytestosterone (for example the back of the head) to a sensitive site where hair has been lost. This autograft approach is limited by the number of hair follicles which can be harvested for redistribution and the cosmetic results are not always consistent but may result in “doll-like hair”.

In other work, chimaeric hair has been generated by grafting tissue containing inductive dermal papilla or dermal sheath cells from a donor into the epidermis of a non-donor recipient (see for example WO01/32840). Such chimaeric hair tends to grow in variable directions and the method does not result in natural-looking hair.

Attempts have been made to inject donor cells into a recipient using a conventional hypodermic needle and syringe. The method does not allow cells to be delivered reproducibly or in controlled amounts into a subcutaneous compartment at an appropriate depth from the surface of the epidermis. Hair follicles induced with the bulb too close to the epidermal/dermal junction are susceptible to being pulled out when placed under a mechanical stress such as combing or brushing. Reproducibly obtaining the correct angle of hair shaft growth has also not been possible. In another approach (see WO02/060396), a “bleb” is formed between the epidermal and dermal layers of the skin by injecting a liquid to create a pocket into which cells can be injected.

A further method for reproduction of hair has been suggested in WO98/47471.. The method involves removing hair in the anagen phase from a donor, culturing the hair follicle cells or keratinocytes in a culture medium and implanting the cultured hair follicle cells into pores of the receptor region. The method proposes to inject the cultured hair follicle cells into existing pores, optionally using a repeating-injection metering device. It remains unclear whether this approach will yield cosmetically useful hair restoration.

The prior art methods of treating baldness are therefore not optimal. The methods are restricted by the inability to control factors such as the density, orientation and positioning of induced or transplanted hair follicles. Furthermore, mechanical techniques (grafting, transplantation and injection) tend to be painful.

In the field of drug delivery, controlled delivery devices have provided safer, more reliable and more effective delivery of fluid drugs than the conventional hypodermic needle and syringe. Examples of such improvements are one or more high velocity driven needles (see WO00/09184), a high pressure fluid delivery system (see U.S. Pat. No. 5,540,657 and U.S. Pat. No. 6,224,567), a tracked injection needle (see U.S. Pat. No. 5,620,421), or needleless delivery means (see US20010027293 A1).

The present inventors have established that, unexpectedly, controlled delivery devices can be utilised to deliver appropriate cells directly into a recipient to provide effective treatments. In particular, controlled delivery devices are found to be useful for treating baldness.

According to the present invention there is provided a method for inducing hair follicle formation in a dermal layer lying beneath an outer skin surface, comprising delivering inductive dermal sheath cells and/or inductive dermal papilla cells directly into the dermal layer using a controlled delivery device.

The present method, particularly as a cosmetic method, is advantageous in that a physician or other practitioner will be able to repeatedly, accurately and precisely deliver cells in a cell suspension or bolus into a subcutaneous compartment. The method allows viable cells to be delivered in a reproducible volume of cell suspension while minimizing the amount of pain experienced by the patient. The method thus provides a significant improvement over prior art methods of treating baldness, in particular over using a traditional hypodermic needle and syringe and over other mechanical techniques such as grafting.

In one aspect, the invention provides a method for inducing hair follicle formation in a dermal layer beneath an outer skin surface, comprising the steps of removing a biopsy containing hair follicles, isolating inductive dermal sheath cells and/or inductive dermal papilla cells from the hair follicles by micro-dissection, expanding the inductive dermal sheath cells and/or inductive dermal papilla cells in culture under conditions which maintain their hair inductive phenotype, and delivering inductive dermal sheath cells and/or inductive dermal papilla cells directly into the dermal layer using a controlled delivery device.

The inductive dermal sheath cells and/or inductive dermal papilla cells do not need to be delivered into pre-existing pores. The invention described in WO98/47471 involves injecting mainly keratinocytes into pre-existing pores. Unlike the present invention, the method of WO98/47471 does not require induction and de novo formation of a new follicle. In the present invention, injected cultured dermal papilla or dermal sheath cells induce the development of new hair follicles, which requires cross-talk with the patient's host keratinocytes. Furthermore, in WO98/47471, when the words “hair follicle cells” are used the implication is clear that the reference is to keratinocytes and not to other cell types in the hair follicle. In paragraph 3 of the description, hair follicle cells are alternatively described as keratinocytes (“hair follicle cells or keratinocytes”) and they are described as cells that convert into the “tough and resilient material which is known as hair”, a reference to the hair shaft that is comprised completely of keratinocytes. In paragraph 4, reference is made that “hair follicle cells . . . form a differentiated epidermis or a fully developed epidermis . . . .” The epidermis does not contain any dermal papillae or sheath cells, so hair follicle cells that form a differentiated epidermis can only be keratinocytes. Another indication of the clear intent that “hair follicle cells” means “keratinocytes” is the use of serum-free keratinocyte culture medium as an example of media available for the growth in culture of “hair follicle cells”. Furthermore, the source of the cultured hair follicle cells is specified to be plucked hairs, which do not normally contain any of the dermal component of hair. Indeed, plucking of hairs by, for example, tape stripping is an experimental method of inducing the anagen phase of the hair cycle, which cannot take place without an intact dermal papilla.

The controlled delivery device may deliver inductive dermal sheath and/or inductive dermal papilla cells in small volumes repeatably, rapidly and consistently. The inductive dermal sheath and/or inductive dermal papilla cells may be delivered in a volume between 0.5 μl and 10 μl, preferably between 1 μl and 2 μl. An issue with existing hair transplantation procedures is the length of time it takes a hair surgeon to place a follicle into the patient's scalp. Typically, the surgeon also requires the assistance of technicians who will harvest the follicles by dissection from the donor biopsy. Once the follicles are dissected, the bald area is sterilised, an incision is made with a scalpel blade, and the donor hair follicle is implanted. In the prior art, a surgeon can typically implant approximately 500 follicles per hour. It is envisaged that the controlled delivery device of the present method in one aspect delivers significant advantages in terms of time for the hair surgeon, due to the reproducible and consistent delivery of small volumes (preferably 1-2 μl of viable inductive DP or DS cells).

The cells preferably remain viable after delivery. For example, loading the cells into the cell delivery device may be performed carefully so as to maintain cell viability. Cells may be sensitive to shear forces when they are forced through the needle of any device, so this step may be monitored also.

Furthermore, the cells used for implanting need to retain their hair inductive capacity. A number of approaches are known including culture with keratinocyte conditioned medium.

The controlled delivery device may be sterilisable if it is to be used on more than one patient to prevent cross contamination of infectious agents from dermal papilla or dermal sheath cells from previous surgical procedures. Typically sterilisation can be achieved by autoclaving. In specific embodiments described below, the PB-600 repeating dispenser of a modified Hamilton syringe is sterilisable, as is the glass syringe barrel. The novel device described in Example 7 is fully steam sterilisable. The controlled delivery device is preferably constructed from non-corrodable materials. For example, surgical grade stainless steel is preferable to aluminium. Alternatively, components which will corrode need to be sufficiently cheap to be single patient use or disposable.

The inductive dermal sheath cells and/or inductive dermal papilla cells may be derived from a variety of sources. One source is mesenchymal stem cells derived from bone marrow (available from Osiris, for example). Another source is bone marrow mesodermal progenitor cells (see WO01/11011—Catharine Verfaillie's multipotent adult progenitor cells). Yet a further source is hematopoietic stem cells derived from human bone marrow. Another source of cells are pluripotent cells derived from the skin (Toma, J. G. et al., 2001, Nature Cell Biol. 3: 778-784; Aegera Therapeutices Inc. & Curis Inc [both US]). Alternatively, the inductive dermal sheath cells and/or inductive dermal papilla cells may be derived from embryonic stem cells. Another source is embryonic carcinoma cells which have been suitably differentiated towards a DP phenotype for hair using known methods. (Teratomas from which embryonic carcinoma cell lines can be derived have hair and teeth-like structures: embryonic carcinoma cells are commercially available from Layton Biosciences [US], for example.) A further source is reprogrammed cells, for example, autologous cells such fibroblasts which have been “reprogrammed” by dermal papilla cells or embryonic carcinoma cells to induce hair formation (for reprogramming of cells, see WO00/49138).

Cells with a desired functionality of hair inducibility may be stably maintained in culture using known methods (see for example: U.S. Pat. No. 5,851,831, for long term subculture of dermal papilla cells; and the methods disclosed in WO01/74164).

The inductive dermal sheath cells and/or inductive dermal papilla cells may be delivered to a depth from an outer surface where normal hair follicles form in vitro (e.g. in cultured skin) or in vivo (i.e. in a person or other mammal). For example, the depth may be 0.5-4.0 mm into human tissue. The controlled delivery device allows the depth of delivery to be precisely determined and consistently reproduced, but is also adjustable for a particular delivery situation. Delivery of the inductive cells to the correct depth allows induced hair follicles to be imbedded in the dermis so that developed hairs will be better anchored and less susceptible to mechanical stresses such as pulling, combing or brushing.

The inductive dermal sheath cells and/or inductive dermal papilla cells may be delivered at a given angle within the dermal layer.

A growing hair follicle will not necessarily automatically orientate itself properly. According to the invention the inductive dermal sheath cells and/or inductive dermal papilla cells may be delivered in a track (or channel) formed by the controlled delivery device and oriented towards an outer surface. The track may be contiguous with the host epidermis. A track provides a pathway which allows a nascent hair follicle to grow in the correct direction towards the surface of the skin and connect with the surface epidermis surrounding the track. In addition, the angle of the track can be varied, allowing the nascent hair follicle to grow at an appropriate angle relative to the outer surface. This achieves a good cosmetic result because hair follicles grow at different angles in different regions of the scalp and a more robust hair follicle. In one embodiment, the controlled delivery device used to generate the track has a needle with blunt end and an orifice on the lateral side near the tip (for example, an orifice approximately 0.5 mm from the tip), allowing cells to be implanted along the needle track.

The controlled delivery device may dispense in suspension between 1000 and 40000 inductive dermal sheath cells and/or inductive dermal papilla cells per delivery. The controlled delivery device may dispense inductive dermal sheath cells and/or inductive dermal papilla cells in suspension at a cell density of between 5×10⁵ and 4×10⁷ cells/ml per delivery. In the specific embodiments described below, experiments were performed to determine the effect of these various parameters.

The controlled delivery device may dispense pre-formed aggregates of inductive dermal sheath cells and/or inductive dermal papilla cells. For example, one to three pre-formed aggregates may be dispensed per delivery. Each pre-formed aggregates may contain between 1.5×10³ and 1×10⁴ cells. In these embodiment, cells are allowed to aggregate, or inducing cells to aggregate, into pre-formed aggregates, i.e. clumps of cells of the appropriate volume and cell number, before being injected subcutaneously. Cells or aggregates of cells may be placed in a formulation, such as hyaluronic acid or glycosaminoglycans, that includes a substance (or substances) which increases the viscosity of the injected material in order to protect the cells during handling and injection. Cells or aggregates of cells may be placed in a formulation, for example one including fibrin, fibronectin and/or collagen or other extra-cellular matrix molecules known to those skilled in the art, that enhances the microenvironment of cells after implantation, in order to facilitate cell migration or cell-cell interaction.

The inductive dermal sheath cells and/or inductive dermal papilla cells may be from a source autologous or allogeneic to the dermal layer.

Preferably, the controlled delivery device comprises one or more high velocity driven needles (for example as claimed in WO00/09184), a high pressure fluid delivery system (for example as claimed in U.S. Pat. No. 5,540,657 or U.S. Pat. No. 6,224,567), a tracked injection needle (for example as claimed in U.S. Pat. No. 5,620,421) or is needleless (for example as claimed in US20010027293 A1).

Alternatively, the controlled delivery device may be a Hamilton syringe with a controlled volume delivery modification (for example, a Hamilton PB-600 repeating dispenser).

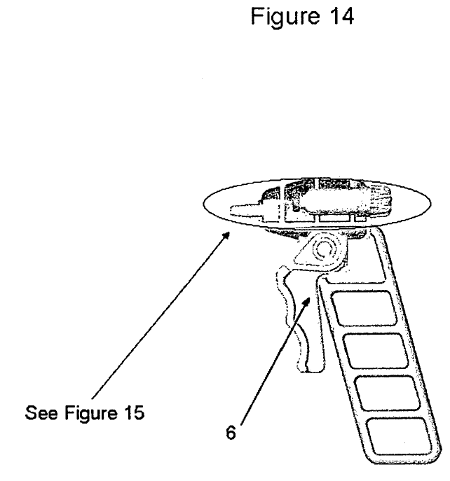

In a further embodiment, the controlled delivery device comprises a micropump dispensing mechanism. Certain prior art devices, such as medication delivery pens, have been developed to improve upon the traditional syringe injection delivery system. These devices are repeatable and typically use a piston mechanism similar to a syringe. However, unlike a typical syringe, an actuator mechanism is used to exert a defined axial force on the piston to inject only the set dosage of medication. However, these devices may be unable to repeatedly inject quantities smaller than 50 μL and to control the depth of injection. However, a controlled delivery device with a micropump dispensing mechanism, as used previously in the microfluidics field to create, for example, micro-sized bioassay systems (“lab-on-a-chip”), cell microarrays and miniaturized chemical analysis systems, may be used in the present invention for delivering a viable cell suspension or cluster of cells. Preferably the controlled delivery device with a micropump dispensing mechanism provides at least one of the following: (i) the ability to deliver minute (<10 μL) amounts of cell suspension, (ii) the ability to deliver such amounts in a repeatable fashion so as to facilitate multiple injections and to avoid the need to refill the device numerous times, and (iii) the ability to determine and control the depth of the injection so as to ensure the proper delivery of cells to a desired injection site. A preferred specific embodiment is shown in FIGS. 14-16 and described further below.

Further provided according to the present invention is a method as described herein additionally including one or more steps resulting in the development of a mature hair follicle.

Also provided according to the present invention is the use of a controlled delivery device for the delivery of inductive dermal sheath cells and/or inductive dermal papilla cells into a dermal layer to induce hair follicle formation. The features pertaining to the method elaborated herein are also applicable for this use.

It will also be appreciated that the invention described herein may be more generally applicable so as to provide a method for inducing organ or tissue formation. The method may comprise delivering inductive cells into an organ or tissue regenerative cellular environment using a controlled delivery device. Furthermore, the invention covers the use of a controlled delivery device for the delivery of inductive cells into a regenerative cellular environment to induce organ or tissue formation. Using a controlled delivery device also allows for cells to be delivered to a specific location for therapeutic purposes.

Embodiments of the invention are now described by way of example with reference to the accompanying drawings in which:

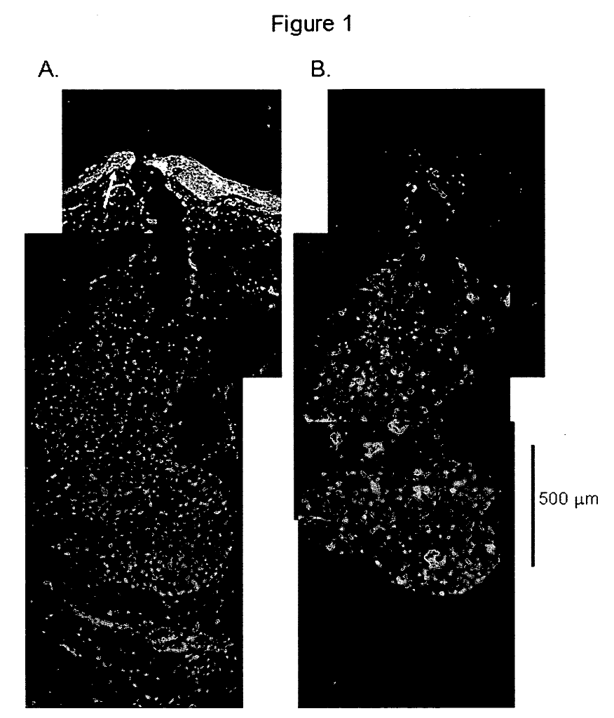

FIG. 1 Shows the injection of DiI-labelled human DP cells delivered to a depth of 1.0 mm into human scalp skin as described in Example 1. Panel A shows fluorescent Hoechst staining of the recipient skin. Panel B shows the bolus of DiI-labelled human DP cells that have been delivered to a depth just below the DEJ (Dermal-Epidermal Junction).

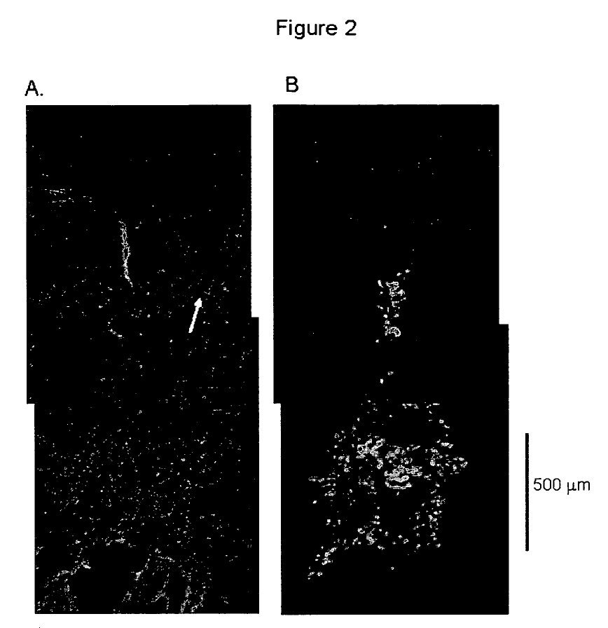

FIG. 2 Shows the injection of DiI-labelled human DP cells delivered to a depth of 1.0 mm into human scalp skin as described in example 1. Panel A shows fluorescent Hoechst staining of the recipient skin. Panel B shows the bolus of delivered DiI-labelled human DP cells. Note that although the major bolus of cells is below the DEJ, in this instance some DP cells remain in the needle track.

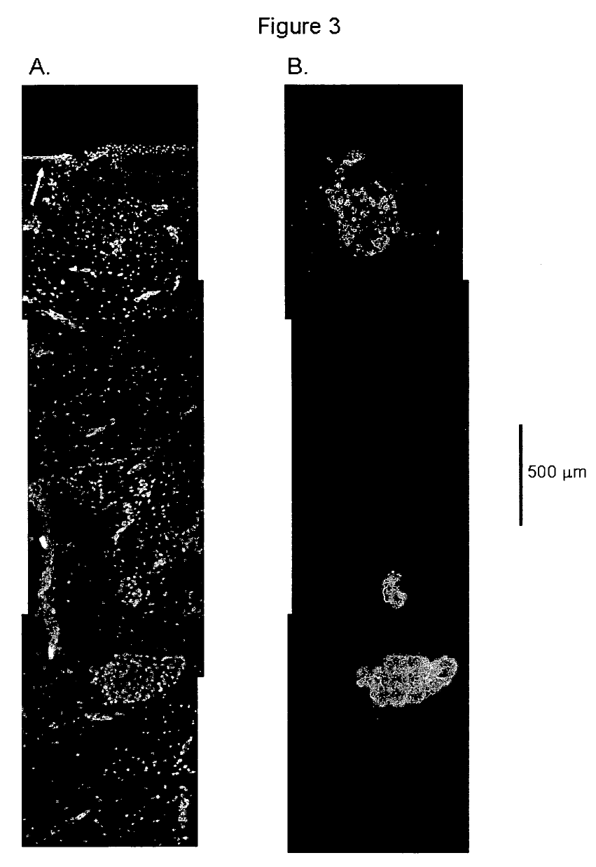

FIG. 3 Shows Composite images of the injection of DiI-labelled human DP cells delivered to a depth of 2.0 mm into human scalp skin as described in Example 1. The photographs are shown to the same scale. Panel A shows fluorescent Hoechst staining of the recipient skin. Panel B shows the bolus of delivered DiI-labelled human DP cells. Note that although the major bolus of cells is below the DEJ, in this instance some DP cells remain near the top of the needle track. This is a common problem and requires good design if in a commercial process valuable DP cells are not to be wasted.

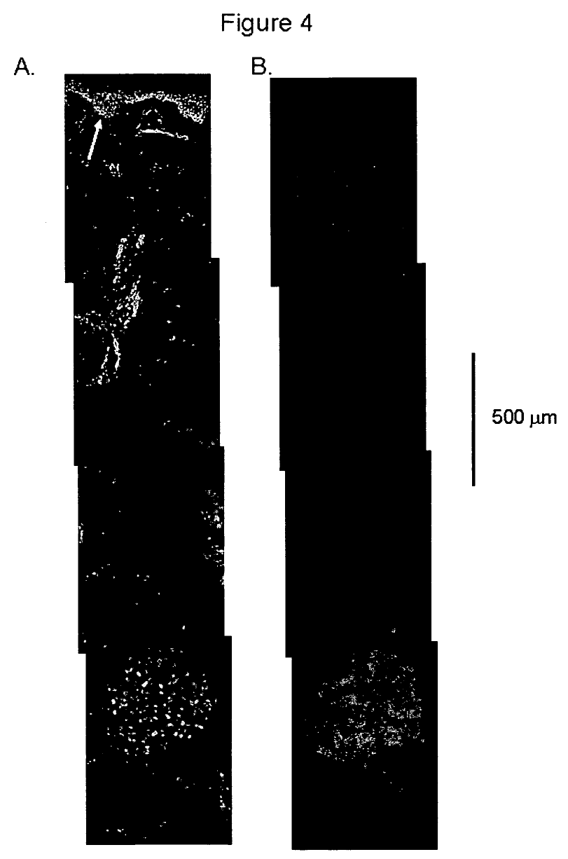

FIG. 4 Shows the injection of DiI-labelled human DP cells delivered to a depth of 2.0 mm into human scalp skin as described in Example 1. The photographs are shown to the same scale. Panel A shows fluorescent Hoechst staining of the recipient skin. Panel B shows the bolus of delivered DiI-labelled human DP cells. Note that the major bolus of labelled DP cells is below the DEJ (note how much deeper in FIGS. 3 and 4 than in FIG. 2 demonstrating the importance of depth control).

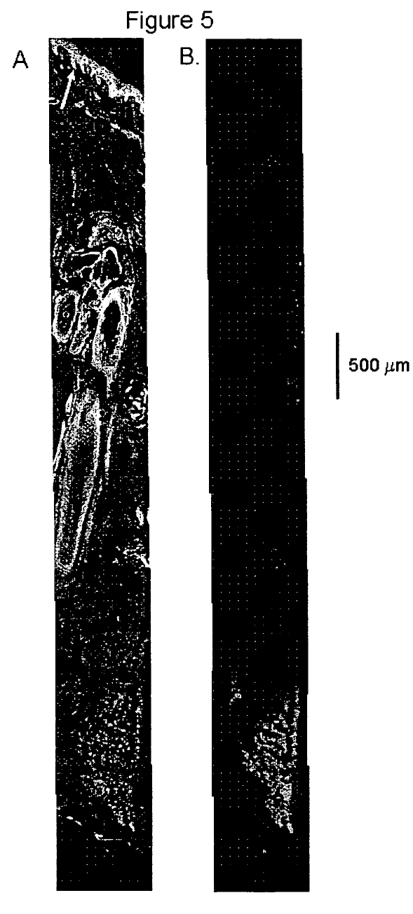

FIG. 5 The injection of DiI-labelled human DP cells delivered to a depth of 4.0 mm into human scalp skin as described in Example 1. The photographs are shown to the same scale. Panel A shows fluorescent Hoechst staining of the recipient skin. Panel B shows the bolus of delivered DiI-labelled human DP cells. Note that the major bolus of labelled DP cells is now significantly below the DEJ (cf. FIGS. 2,3,4).

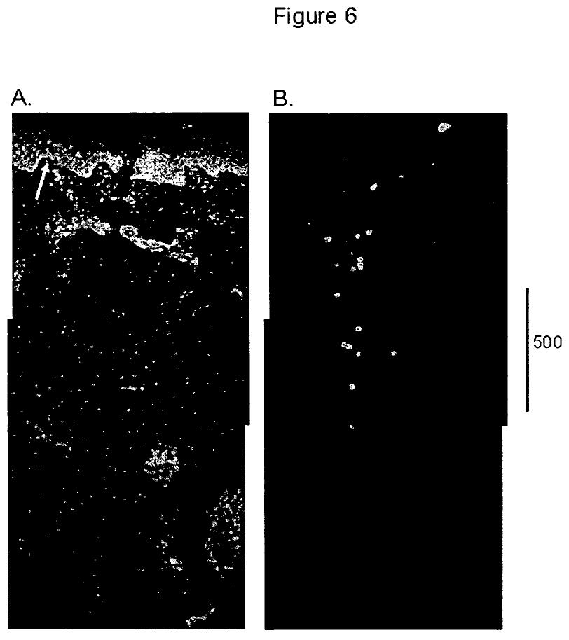

FIG. 6 Shows attempted delivery of 1×10³ human DP cells ex vivo into porcine skin to a depth of 1.0 mm as described in Example 2. Panel A is Hoechst staining of the skin. Panel B visualises DiI-labelled human DP cells. Note the low efficiency of cell delivery. At this cell density, it is difficult to deliver a consistent bolus, and delivery is highly variable. The invention preferably uses higher concentrations of delivered DP cells.

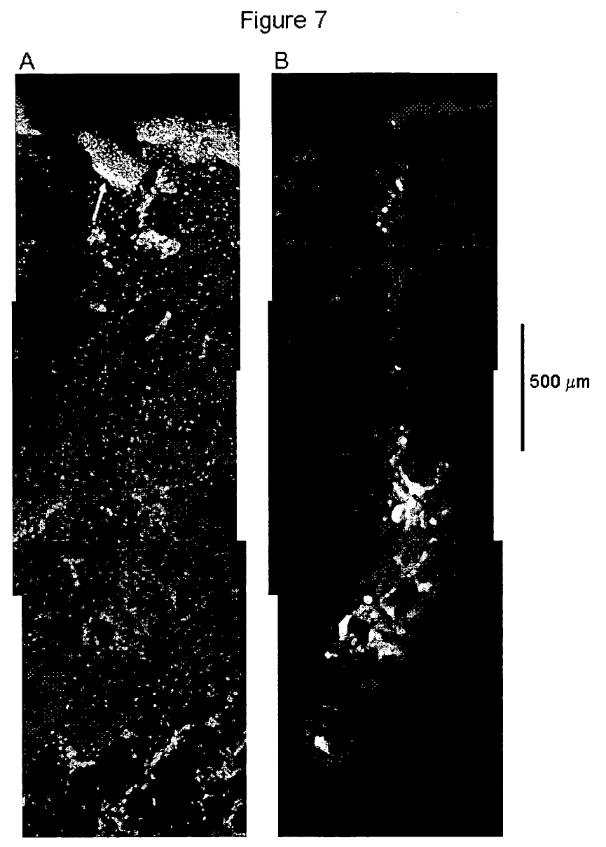

FIG. 7 Shows delivery of 1×10⁴ human DP cells ex vivo into porcine skin to depth of 1.0 mm as described in Example 2. Panel A shows Hoechst staining of the skin, while Panel B shows DiI-labelled human DP cells. Compared with FIG. 6, this higher cell density delivers a more consistent and discreet bolus. In one embodiment, the invention therefore preferably uses concentrations of delivered DP cells around this range.

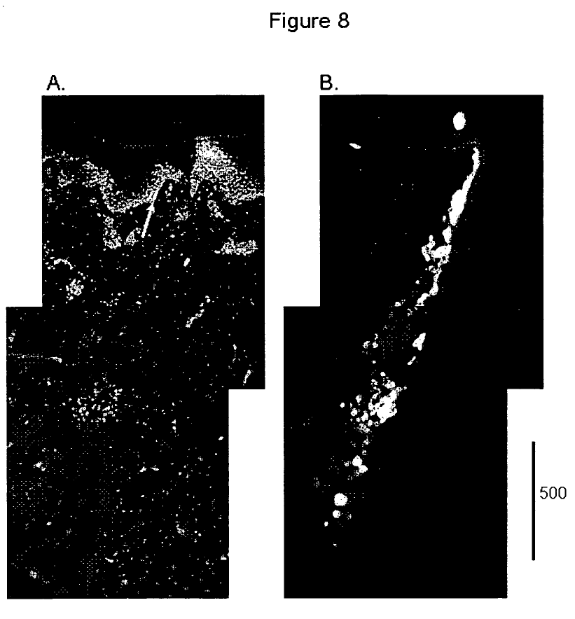

FIG. 8 Shows delivery of 4×10⁴ human DP cells in a 1.0 μl volume (4×10⁷ cells/ml) ex vivo into porcine skin to depth of 1.0 mm as described in Example 2. Panel A shows Hoechst staining of the skin, while Panel B visualises DiI-labelled human DP cells. Note that at this higher cell density than in FIG. 7, delivery of a bolus is still consistent and discreet. In a further embodiment, the invention preferably also uses concentrations of delivered DP cells around this range.

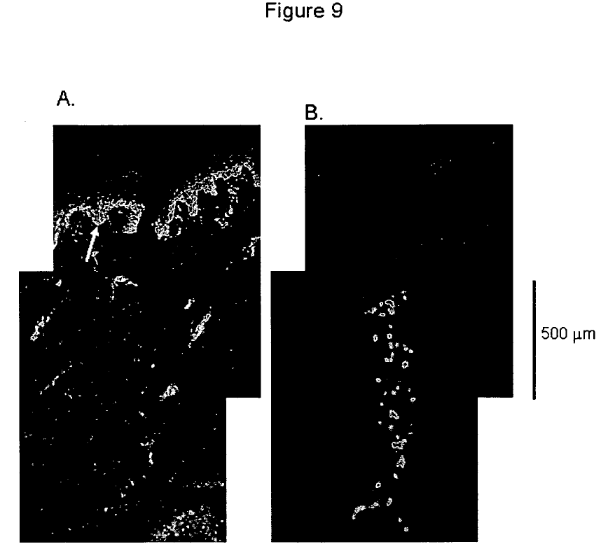

FIG. 9 Shows delivery of 2×10⁴ human DP cells in a 0.5 μl volume (4×10⁷ cells/ml) ex vivo into porcine skin to a depth of 1.0 mm as described in Example 3. Panel A shows Hoechst staining of the skin, while Panel B visualises DiI-labelled human DP cells. The delivery of DP cells under these conditions is sub-optimal and few cells remain in the needle track.

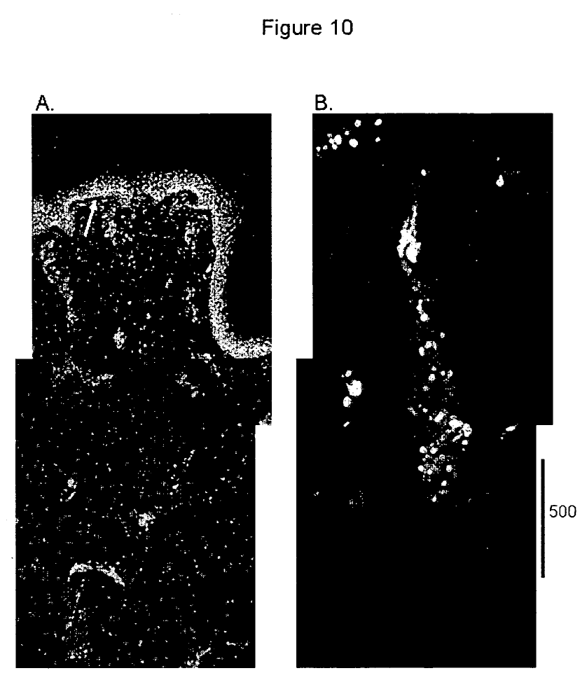

FIG. 10 Shows delivery of 4×10⁴ human DP cells in a 1.0 μl volume (4×10⁷ cells/ml) ex vivo into porcine skin to depth of 1.0 mm as described in Example 3. Panel A shows Hoechst staining of the skin, while Panel B visualises DiI-labelled human DP cells. The delivery of DP cells under these conditions is more consistent and many cells remain in the needle track.

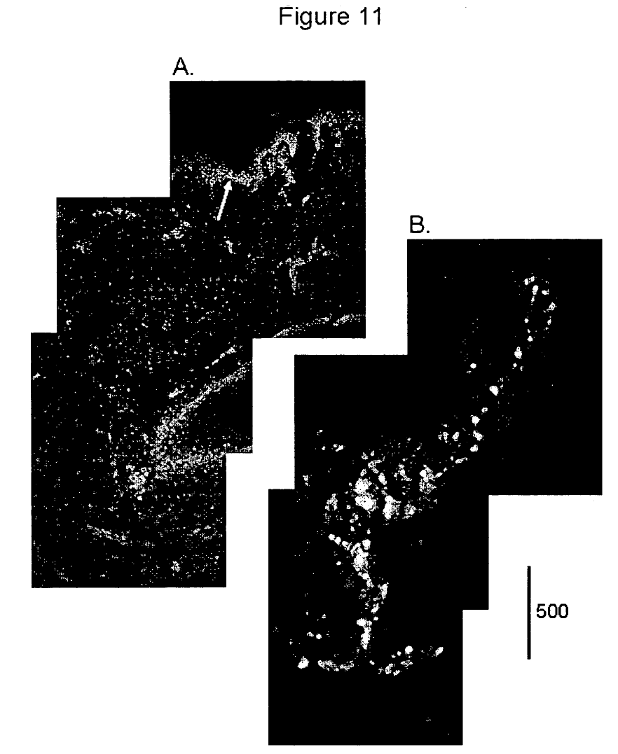

FIG. 11 Shows delivery of 8×10⁴ human DP cells in a 2.0 μl volume (4×10⁷ cells/ml) ex vivo into porcine skin to depth of 1.0 mm as described in Example 3. Panel A shows Hoechst staining of the skin, while Panel B visualises DiI-labelled human DP cells. The delivery of DP cells under these conditions is more consistent and many cells remain in the needle tracks.

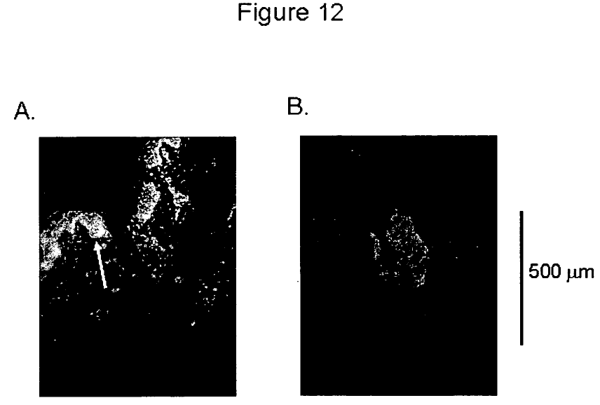

FIG. 12 Shows delivery of a pre-formed aggregate of 2×10³ human DP cells beneath the DEJ of porcine skin as described in Example 4.

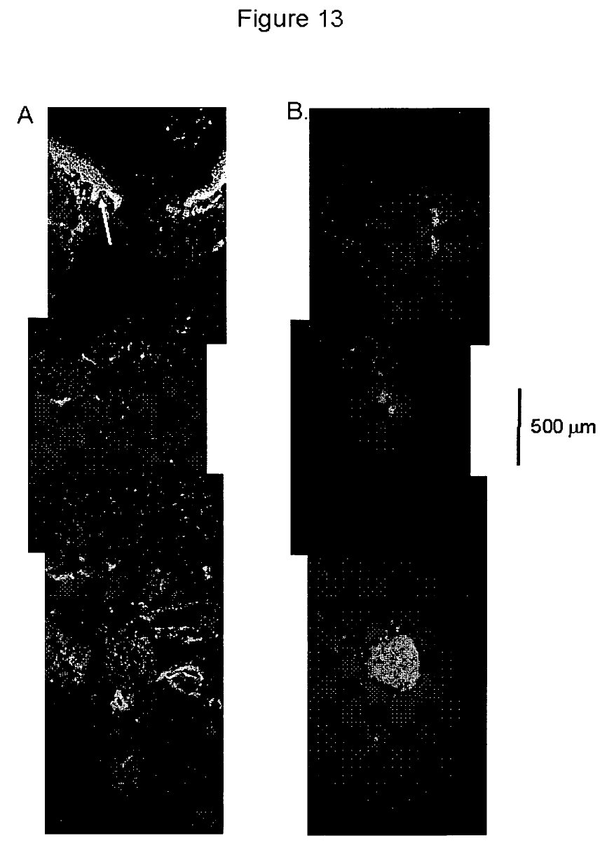

FIG. 13 Shows delivery into porcine skin of a pre-formed aggregate of 2×10³ human DP cells to the depth where normal dermal papilla are located, as described in Example 4.

FIG. 14 Shows a micropump injector device for use in delivery of cells;

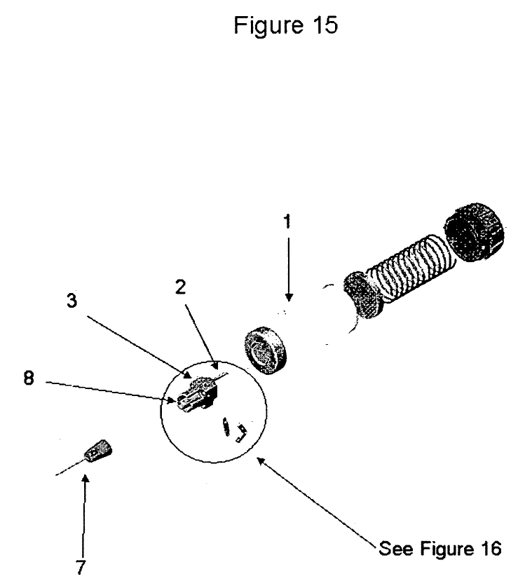

FIG. 15 Shows an enlarged view of a portion of the micropump injector device as shown in FIG. 14.

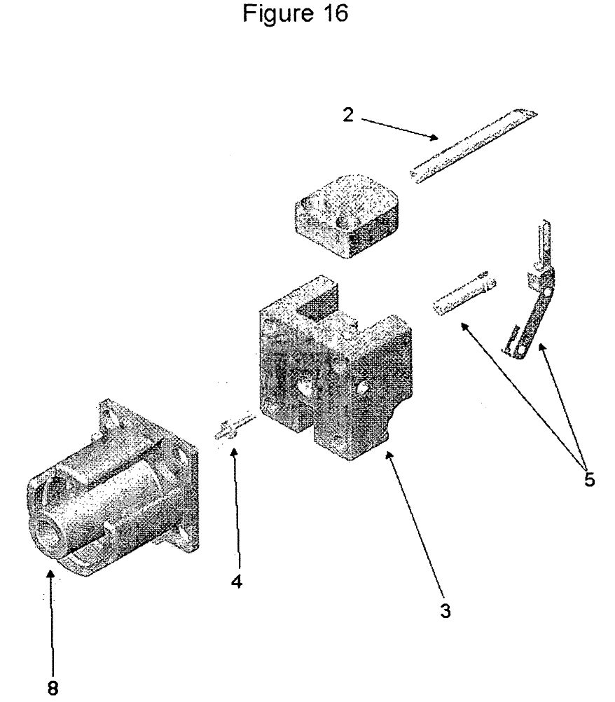

FIG. 16 Shows an enlarged view of the portion of the micropump injector device shown in FIG. 15.

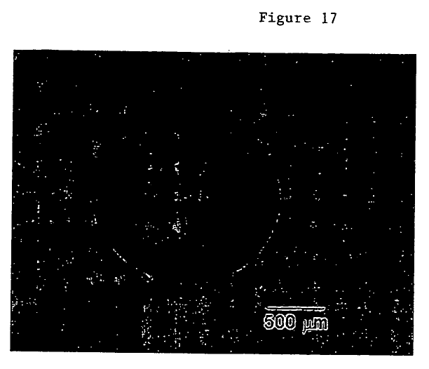

FIG. 17 Shows pre-formed-DiI labelled cell aggregates constituted from passage 2 cultured human DP cells before injection as described in Example 4. The results of injection of these aggregates are shown in FIGS. 12 and 13.

In all the histology figures stained with Hoeschst (FIGS. 1-13) the epidermal layer stains brighter (due to the presence of a higher density of nuclei) than the dermal layer. The Dermal-Epidermal junction (DEJ) is highlighted with an arrow.

### EXPERIMENTAL

The present invention discloses methods for the controlled delivery of small volumes of liquid containing inductive DP or DS cells to a position below the dermal-epidermal junction (DEJ). To demonstrate that a variety of different controlled delivery devices can be used to deliver DP cells, we provide examples using a Hamilton syringe with a controlled volume delivery modification, a high velocity injection device, and a novel design for a controlled delivery device.

### Example 1

### Demonstration of Controlled Delivery of Cells Into Human Skin ex vivo: Effect of Depth of Delivery

To test the effect of the depth of delivery of cells using the Hamilton syringe with the controlled volume modification, human DP cells were injected into human scalp skin biopsies ex vivo.

Using fine forceps and a scalpel under stereomicroscopic observation, intact human hair follicles were individually dissected from a small piece of scalp biopsy. Each individual follicle was further dissected to remove the lower dermal sheath and dermal papilla from the epidermal portion of the follicle. The dermal papillae were separated from the dermal sheathes using a hypodermic needle as a scalpel and were then placed in culture medium. The medium used for propagation of dermal sheath or dermal papilla cells was Chang Medium (Irvine Scientific, Santa Ana, Calif.) combined with keratinocyte conditioned medium similar to that described by Matsuzaki (Hair Research for the Next Millenium, Elsevier Science, pp 447-451, 1996). The papillae were allowed to attach and grow to confluence.

Once the cells had reached confluence, they were trypsinized to prepare a single cell suspension and then labeled with the flourescent dye, DiI. A cell suspension of 4×10⁷ cells/ml was prepared and placed in a sterile 1 mL vial. A 50 μL Hamilton syringe attached to a Hamilton PB-600 repeating dispenser and a 27G-½″ needle was then filled with the cell suspension. The repeating dispenser allows for 50 incremental dispenses, resulting in 50-1 μL injections that deliver 4×10⁴ human DP cells each. A sterile, polyethylene ring was also placed around the needle to act as a “stop” to control the depth of the injection to 1 mm (FIGS. 1-2), 2 mm (FIGS. 3-4) or 4 mm (FIG. 5).

Dermal papilla cells were injected at controlled depths into full thickness human scalp biopsies. Immediately after injection, skin samples were fixed in 10% formalin overnight and then embedded in tissue embedding media for cryosectioning and histology. Ten to twenty μm thick sections of tissue were cut, placed on glass slides and stained with fluorescent Hoechst stain in order to view the nuclei of all cells in the tissue (FIGS. 1A, 2A, 3A, 4A, 5A).

Stained sections were viewed under fluorescent microscopy to verify placement and depth of the injected DP cells (FIGS. 1B, 2B, 3B, 4B, 5B). Injections made to a depth of 1 mm resulted in the placement of DP cells in contact with the epidermal-dermal junction (FIGS. 1-2). Injections at 2 mm and 4 mm resulted in placement of cells at depths where the dermal papilla of normal human hair follicles typically reside (FIGS. 3-5).

### Example 2

### Demonstration of Controlled Delivery of Cells into Porcine Skin ex vivo: Effect of Cell Number

The effect of cell number on the delivery of DP cells using the Hamilton syringe with the controlled delivery modification was tested using porcine skin as a model for human skin.

Dermal papilla cells were isolated, grown in culture and stained with DiI as in Example 1. Cell suspensions were prepared at concentrations of 1×10⁶, 1×10⁷ and 1×10⁸ cells/mL. Each cell suspension was loaded into a separate 50 μL Hamilton syringe attached to a Hamilton PB-600 repeating dispenser and a 27G-½ needle. One microliter volumes (1000, 1×10⁴, and 1×10⁵ cells/injection, respectively) were injected with each cell concentration to a depth of 1 mm. Immediately following injection, tissue samples were fixed, embedded, sectioned and stained as in Example 1.

At 1×10⁵ cells/injection, the cell suspension was too viscous to be consistently delivered through a 27G needle. At 1×10³ cells/injection, cells were too disperse and did not consistently result in a discreet bolus of cells at the defined depth (FIG. 6). Consistent results were obtained with delivery at 1×10⁴ cells/injection (FIG. 7). This consistency could be sustained at a cell concentration up to 8×10⁴ cells/injection (4×10⁷ cells/ml) (FIGS. 8, 10).

### Example 3

### Demonstration of Controlled Delivery of Cells into Porcine Skin ex vivo: Effect of Volume

The effect of volume on the delivery of DP cells using the Hamilton syringe with the controlled delivery modification was tested by injecting cultured DP cells into full thickness porcine skin ex vivo.

Dermal papilla cells were cultured and prepared as previously stated in Examples 1 and 2. A cell concentration of 4×10⁷ cells/mL was prepared and loaded into either a 25 μl or 50 μl Hamilton syringe with Hamilton PB-600 repeating dispenser and a 27G-½″ needle. Use of a 25 μl syringe resulted in a delivery volume of 0.5 μl, while a 50 μl syringe results in a 1 μl delivery. A 2 μl delivery volume was obtained by delivering 2 dispenses into the same injection using a 50 μl syringe. Immediately following injection, tissue samples were fixed, embedded, sectioned and stained as in Example 1.

When delivered at a volume of 0.5 μl, few cells remained in the injection track after delivery (FIG. 9). Consistent delivery of cells into the injection track was achieved with both 1 μl and 2 μl delivery volumes (FIGS. 10, 11).

### Example 4

### Controlled Delivery of Cell Aggregates into Porcine Skin ex vivo

Dermal papilla cells were isolated and expanded in culture as described in Example 1. After two passages, confluent cells were trypsinized, stained with fluorescent DiI, and suspended in culture medium at a concentration of 2×10⁴ cells/mi. Aliquots of 100 μL of cell suspension (2×10³ cells) were placed in sterile 0.5 mL microcentrifuge tubes and centrifuged at approximately 200 g for 5 minutes to pellet the cells. Tubes were then incubated overnight at 37° C. The following day, DP cell aggregates were removed from the tubes using a 50 μl micropipette, collected in a fresh, sterile 1.0 ml microcentrifuge tube and centrifuged again at 200 g for 5 minutes. Excess medium was removed and fresh medium was added to make a suspension of 3 aggregates/μl. This concentration of aggregates resulted in 1-3 aggregates being delivered per 1 μl injection. A 50 μl Hamilton syringe attached to a Hamilton PB-600 repeating dispenser and an 18G needle was loaded with the aggregate suspension. Controlled, 1 μl volumes of aggregate solution were injected into full-thickness porcine skin.

As in Example 1, tissue samples were fixed in 10% formalin immediately following injection and embedded in tissue embedding media for cryosectioning and staining with Hoechst stain. Depending on the depth of injection, aggregates could be placed at the epidermal-dermal junction (FIG. 12) or at the depth of normal dermal papilla (FIG. 13).

### Example 5

### Delivery of Inductive Autologous Human DP Cells into Human Volunteers using a Hamilton Syringe with Controlled Volume Modification

Dermal papilla cells are isolated from fresh dermal papilla dissected from a full thickness biopsy of human scalp skin from human volunteers under informed consent. Cells are then expanded in tissue culture. Cells are removed from the culture dishes via trypsinization, and a cell suspension of 1-4×10⁷ cells/ml is prepared and placed in sterile 0.5 ml vials. As in Example 1, a 50 μl Hamilton syringe attached to a Hamilton PB-600 repeating dispenser and a 27G-½″ needle is loaded with the cell suspension. Injections of 1 μl each are made into the scalp of each volunteer.

### Example 6

### Delivery of Inductive Autologous Porcine DP Cells using Hamilton Syringe with a Controlled Volume Modification

Dermal papilla (DP) cells are isolated from fresh dermal papilla dissected from a full thickness biopsy of porcine skin taken from an area having a high density of hair. Cells are expanded in culture as described in Example 1. When confluent, the porcine DP cells are removed from the culture dishes via trypsinization and a cell concentration suspension of 1-4×10⁷ cells/ml is prepared and placed in sterile 0.5 ml vials.

Three healthy hybrid, female or castrated male pigs, 2-5 months old, are used in this study. An area of the scalp with a low density of hairs, ideally hairless, is selected and marked for injection of the DP cells. Local anesthesia is achieved by injecting 0.5-1.0% lignocaine with 1:200,000 adrenaline into the scalp surrounding the treatment area. A prick test is performed to ensure numbness and 20 mg or 10 mg Temazepam is given orally before treatment in order to relax the animal. A Hamilton syringe with a PB-600 repeating dispenser and 27G-½″ needle, as described in Example 1, is loaded and used to make injections into the treatment area.

DP Cells are injected at depths of 1 mm, 2 mm, or 4 mm. In addition, at each injection depth, injections are made an angle from the vertical of 0°, 45°, or 90°. Wounds are covered with OpSite bandages to minimize desiccation and the chance of infection. Post-operatively, the animal is given 1-2 tablets of Coproximol every 4-6 hours for pain and 40 mg reduced to 20 mg Predinisolone over 5 days.

The area of injection is monitored using macro photography and 4× magnification loupes. Photographs are taken prior to implantation, immediately after implantation, and 28 days post implantation. In addition, biopsies are taken at 28 days and processed using routine histological paraffin techniques for staining with haematoxylin and eosin. All biopsies are assessed for the presence of hair, the presence and density of DP cells and any evidence of inflammation or angiogenesis within or adjacent to the injection sites.

### Example 7

### Novel Controlled Delivery Device

The majority of the current injection devices operate based on the mechanics of the traditional piston syringe. That is, they utilize a hollow barrel through which a piston-like plunger acts to aspirate and dispense the injectable material. Current repeatable syringes operate by incrementally advancing the plunger rather than dispensing the entire syringe barrel at once. The limitations in the accuracy of this method of obtaining multiple injections results in the inability of these syringes to accurately dispense multiple microliter amounts without sacrificing the number of possible injections per syringe fill. Thus, the smaller the individual injection one desires, the smaller the overall capacity of the syringe must be, thereby limiting the total number of injections possible per syringe fill. The present invention improves upon these limitations by using a micropump dispensing mechanism rather than the traditional piston-syringe mechanism.

In this embodiment of the invention, the micropump injector device utilizes a cell suspension reservoir containing hair inductive cells which is supplied in a septum-covered, pressurized vial with a spring-loaded bottom (FIG. 15). As illustrated in FIG. 15, this reservoir (1) is inserted into the device such that the septum is punctured by a needle (2) that feeds into the micropump reservoir chamber (3). The micropump itself is illustrated in FIG. 16 and is composed of a small chamber (3) with a passive one-way microvalve (4) and a piston actuation mechanism (5), which, in turn, is mechanically controlled by the user using an actuator trigger (6) illustrated in FIG. 14. A standard luer needle (7) (FIG. 15) is attached to the end of the device via a luer lock (8) (FIGS. 15-16). Depression of the actuator trigger causes the actuation piston to enter the chamber cylinder and open the passive one-way microvalve, forcing air and liquid out of the chamber. Release of the actuator trigger causes the actuator piston to be removed from the chamber and for the passive one-way microvalve to close, resulting in the flow of cell suspension into the chamber from the cell suspension reservoir. With each push of the actuator trigger, therefore, a defined 1 μl amount of cell suspension is released from the chamber reservoir into the attached needle and injected. With the release of the actuator trigger, the micropump chamber is re-filled with cell suspension from the cell suspension reservoir. The overall size of the cell suspension reservoir may be 1 ml, thus allowing for 1000 incremental 1 μl injections.

### Example 8

### Delivery of Inductive Autologus Human DP Cells using a Novel Controlled Delivery Device

A high cell concentration suspension is prepared as in Example 1 and placed in a sterile 1 mL serum bottle which is then septum-sealed. This bottle is then loaded into the mechanically actuated pump device described in Example 7. This is designed to inject 1 μL of cell suspension into the scalp.

### Example 9

### Delivery with High-Velocity Driven Needle Device (Imprint)

A full-thickness piece of human male scalp skin is placed into a small amount of medium. Using fine forceps and scalpel under stereomicroscopic observation, intact hair follicles are individually dissected from the piece of skin. Each individual follicle is further dissected, first to remove excess connective tissue, then to remove the lower dermal sheath and dermal papilla from the epidermal portion of the follicle. Once the sheaths and papillae are separated from the rest of the follicle, the dermal papillae are separated from the sheaths at the stalk that connects the two structures using a hypodermic needle as a scalpel. Dermal papillae are placed into culture medium and allowed to attach. Typically, 2-3 papilla are placed into a well of a 12-well culture dish. After several days, papillae are analysed to detect cells growing out from them. When explants have been grown for a further 10-12 days, cultures are trypsinized and replated at a density of 2-3×10³ cells/cm². After one week, cells are confluent and ready for transplantation. Medium used for propagation of cells is Chang Medium (Irvine Scientific, Santa Ana, Calif.) combined with human keratinocyte conditioned medium.

To prepare cells for transplantation, cells are removed from culture dishes by trypsinisation. A high concentration cell suspension is prepared (10³-10⁴ cells per μl) and then loaded into an high velocity driven needle device (see WO00/09184). The needle of the device has a blunt end and an orifice that is on the lateral side approximately 0.5 mm from the tip, allowing cells to be implanted along the needle track. This allows the implanted cells to be in close proximity to the epidermis and it also facilitates cell migration along the track. The angle of the track determines the angle of hair growth, and the depth ensures that the hair follicle will be well anchored in the dermis.

Full-thickness human female skin from a hairless region of the body (obtained from breast reduction surgery) is grafted on athymic mice anaesthetised with ketamine/xylazine. Using the delivery device, the hair inductive cells are implanted into the grafted skin. The depth of the needle tip is 0.2-0.8 mm below the surface of the skin and depends upon the depth setting of the device and the angle at which the needle is inserted. For each separate injection, a cell suspension volume of 1-5 μl is injected, delivering 2-10×10³ hair inductive cells. After the injections, mice are allowed to recover from the anaesthesia and are housed individually in separate cages. After approximately 6 weeks, the mice are monitored for hair induction at the injection sites.

In general terms, the skilled person should appreciate that the efficiency of the novel organ formation may be dependent on the following factors:
the number of inductive cells injected at a given site;
the exact position and distance of inductive cells with respect to the dermal-epidermal junction (for example the depth of the needle track);
the inductive status of the inductive cells;
the passage number of the inductive cells;
the culture media used in culture for expanding the inductive cells;
how the inductive cells are “aggregated” prior to loading the delivery system (nucleation of aggregates using a number of beads [e.g. microcarrier beads] may be useful); and
the production of a suitable wound in the dermis.
   These factors can be modified to obtain optimal results for a given application.

1. A method for inducing hair follicle formation in a dermal layer lying beneath an outer skin surface, comprising delivering inductive dermal sheath cells and/or inductive dermal papilla cells directly into the dermal layer using a controlled delivery device:

2. A method for inducing hair follicle formation in a dermal layer beneath an outer skin surface, comprising the steps of removing a biopsy containing hair follicles, isolating inductive dermal sheath cells and/or inductive dermal papilla cells from the hair follicles by micro-dissection, expanding the inductive dermal sheath cells and/or inductive dermal papilla cells in culture under conditions which maintain their hair inductive phenotype, and delivering inductive dermal sheath cells and/or inductive dermal papilla cells directly into the dermal layer using a controlled delivery device.

3. The method according to claim 1, wherein the inductive dermal sheath cells and/or inductive dermal papilla cells are not delivered into pre-existing pores.

4. The method according to claim 1, wherein the controlled delivery device delivers inductive dermal sheath and/or inductive dermal papilla cells in small volumes repeatably, rapidly and consistently.

5. The method according to claim 1, wherein the inductive dermal sheath and/or inductive dermal papilla cells are delivered in a volume between 0.5 μl and 10 μl, preferably between 1 μl and 2 μl.

6. The method according to claim 1 wherein the cells remain viable after delivery.

7. The method according to claim 1, wherein the controlled delivery device is sterilisable.

8. The method according to claim 1, wherein the inductive dermal sheath cells and/or inductive dermal papilla cells are derived from mesenchymal stem cells and/or mesodermal progenitor cells and/or hematopoietic stem cells and/or embryonic stem cells and/or embryonic carcinoma cells and/or reprogrammed cells.

9. The method according to claim 1, wherein the inductive dermal sheath cells and/or inductive dermal papilla cells are delivered to a depth from an outer skin surface, the depth corresponding to a position where normal hair follicles form in vitro or in vivo.

10. The method according to claim 9, wherein the depth is 0.5-4.0 mm into human tissue.

11. The method according to claim 1, wherein the inductive dermal sheath cells and/or inductive dermal papilla cells are delivered at a given angle within the dermal layer.

12. The method according to claim 1, wherein the controlled delivery device dispenses in suspension between 1000 and 40000 inductive dermal sheath cells and/or inductive dermal papilla cells per delivery.

13. The method according to claim 1, wherein the controlled delivery device dispenses inductive dermal sheath cells and/or inductive dermal papilla cells in suspension at a cell density of between 5×10⁵ and 4×10⁷ cells/ml per delivery.

14. The method according to claim 1, wherein the controlled delivery device dispenses pre-formed aggregates of inductive dermal sheath cells and/or inductive dermal papilla cells.

15. The method according to claim 14, wherein one to three pre-formed aggregates are dispensed per delivery.

16. The method according to claim 14, wherein each pre-formed aggregates contain between 1.5×10³ and 1×10⁴ cells.

17. The method according to claim 1, wherein the inductive dermal sheath cells and/or inductive dermal papilla cells are from a source autologous or allogeneic to the dermal layer.

18. The method according to claim 1, wherein the controlled delivery device comprises one or more high velocity driven needles, a high pressure fluid delivery system, a tracked injection needle.

19. The method according to any of claim 1, wherein the controlled delivery device is a Hamilton syringe with a controlled volume delivery modification.

20. The method according to any of claim 1, wherein the controlled delivery device comprises a micropump dispensing mechanism.

21. The method according to claim 1, which additionally includes one or more steps resulting in the development of a mature hair follicle.

22. Use of a controlled delivery device for the direct delivery of inductive dermal sheath cells and/or inductive dermal papilla cells into a dermal layer to induce hair follicle formation.

23. The use according to claim 22, wherein the inductive dermal sheath cells and/or inductive dermal papilla cells are not delivered into pre-existing pores.

24. The use according to either of claim 22, wherein the controlled delivery device delivers inductive dermal sheath and/or inductive dermal papilla cells in small volumes repeatably, rapidly and consistently.

25. The use according to any of claim 22, wherein the inductive dermal sheath and/or inductive dermal papilla cells are delivered in a volume between 0.5 μl and 10 μl, preferably between 1 μl and 2 μl.

26. The use according to any of claim 22, wherein the cells remain viable after delivery.

27. The use according to any of claim 22, wherein the controlled delivery device is sterilisable.

28. The use according to any of claim 22, wherein the inductive dermal sheath cells and/or inductive dermal papilla cells are derived from mesenchymal stem cells and/or mesodermal progenitor cells and/or hematopoietic stem cells and/or embryonic stem cells and/or embryonic carcinoma cells and/or reprogrammed cells.

29. The use according to any of claim 22, wherein the inductive dermal sheath cells and/or inductive dermal papilla cells are delivered to a depth an outer skin surface, the depth corresponding to a position where normal hair follicles form in vitro or in vivo.

30. The use according to claim 29, wherein the given depth is 0.5-4.0 mm into human tissue.

31. The use according to any of claim 22, wherein the inductive dermal sheath cells and/or inductive dermal papilla cells are delivered at a given angle within the dermal layer.

32. The use according to any of claim 22, wherein the controlled delivery device dispenses in suspension between 1000 and 40000 inductive dermal sheath cells and/or inductive dermal papilla cells per delivery.

33. The use according to any of claim 22, wherein the controlled delivery device dispenses inductive dermal sheath cells and/or inductive dermal papilla cells in suspension at a cell density of between 5×10⁵ and 4×10⁷ cells/ml per delivery.

34. The use according to any of claim 22, wherein the controlled delivery device dispenses pre-formed aggregates of inductive dermal sheath cells and/or inductive dermal papilla cells.

35. The use according to claim 34, wherein one to three pre-formed aggregates are dispensed per delivery.

36. The use according to either of claim 34, wherein each pre-formed aggregates contain between 1.5×10³ and 1×10⁴ cells.

37. The use according to any of claim 22, wherein the inductive dermal sheath cells and/or inductive dermal papilla cells are from a source autologous or allogeneic to each other and/or the dermal layer.

38. The use according to any of claim 22, wherein the controlled delivery device comprises one or more high velocity driven needles, a high pressure fluid delivery system, a tracked injection needle or is needleless.

39. The use according to any of claim 22, wherein the controlled delivery device is a Hamilton syringe with a controlled volume delivery modification.

40. The use according to any of claim 22, wherein the controlled delivery device comprises a micropump dispensing mechanism.

41. The use of a controlled delivery device for the delivery of inductive cells into a regenerative cellular environment to induce organ or tissue formation.

42. A method for inducing organ or tissue formation, comprising directly delivering inductive cells into an organ or tissue regenerative cellular environment using a controlled delivery device.

43. The method according to claim 32, additionally comprising one or more steps resulting in the development of a functional organ or tissue.
